(19) United States
(12) Patent Application Publication
(10) Pub. No.: US 2024/0216242 A1
(43) Pub. Date: Jul. 4, 2024

(54) COMPOSITION FOR CLEANSING AND/OR REMOVING MAKEUPS FROM KERATIN MATERIALS

(71) Applicant: L'OREAL, Paris (FR)

(72) Inventors: Yong WANG, Shanghai (CN); Chunyue LIU, Shanghai (CN)

(73) Assignee: L'OREAL, Paris (FR)

(21) Appl. No.: 18/558,506

(22) PCT Filed: May 26, 2021
(86) PCT No.: PCT/CN2021/096044

§ 371 Date: Nov. 1, 2023

(51) Int. Cl. A61K 8/44; A61K 8/34; A61K 8/42; A61Q 1/14

(52) U.S. Cl. CPC A61K 8/442; A61K 8/342; A61K 8/42; A61Q 1/14

(57) ABSTRACT

It relates to a composition for cleansing and/or removing makeups from keratin materials, comprising in an aqueous phase: a) at least one amino acid surfactant: b) at least one C12-C24 saturated monoalcohol: and c) at least one divalent and/or trivalent metal salt. It also relates to a non-therapeutic process for cleansing and/or removing makeups from keratin materials, in particular the skin or the hair, comprising the application of the composition to the keratin materials, in particular the skin or the hair, and rinsing off said composition after an optional period of time.

### TECHNICAL FIELD

The present invention relates to a cosmetic composition, in particular, a composition for cleansing and/or removing makeups from keratin materials. The present invention also relates to a non-therapeutic process for cleansing and/or removing makeups from keratin materials using said composition.

### BACKGROUND ART

Cleansing the skin or removing makeups from the skin is very important for caring for the skin. It must be as efficient as possible because greasy residues, such as excess sebum, the remnants of cosmetic products used daily and make-up products, in particular waterproof products, accumulate in the skin folds, and can block the pores of the skin and result in the appearance of spots.

Several types of skin cleansing or makeup removing products, for example, rinsable anhydrous oils and gels, foaming creams and lotions, are known.

Rinsable anhydrous oils and gels have a cleansing or makeup removing action by virtue of oils present in these formulations. These oils make it possible to dissolve fatty residues and to disperse make-up pigments. These products are effective and well tolerated.

However, they exhibit the disadvantages of being heavy, of not foaming and of not conferring a good skin finish upon application, which is disadvantageous from a cosmetic viewpoint.

On the other hand, foaming creams, lotions and gels have a cleansing or makeup removing action by virtue of the surfactants, which suspend the fatty residues on the face. They are effective and pleasant to use because they foam and they are easy to remove.

In addition, for some lotions, there is dripping issue due to the low viscosity. Amino acid surfactants are commonly used in skin cleansing or makeup removing products due to their mildness. However, the rinsibility is not satisfying. Furthermore, for some commercial cleanser, the rinse-off speed is not quick, which brings unpleasant usage.

Therefore, there is a need to formulate compositions for cleansing or removing makeups from keratin materials, which are mild and oil-resistant, do not have a dripping issue, can provide a good foam density, a quick rinse-off speed, and a good skin finish, i.e, a not tight not dry skin feeling.

### SUMMARY OF THE INVENTION

The inventors have found that such a need can be achieved by the present invention.

Thus, according to an aspect, the present invention provides a composition for cleansing and/or removing makeups from keratin materials, comprising in an aqueous phase:
a) at least one amino acid surfactant;
b) at least one C12-C24 saturated monoalcohol; and
c) at least one divalent and/or trivalent metal salt.

The composition according to the present invention presents a paste, cream, or gel texturized lotion aspect.

The composition according to the present invention is mild and oil-resistant, and does not have a dripping issue.

The composition according to the present invention can provide a good foam density, a quick rinse-off speed, and a good skin finish (i.e. a not tight and not dry skin feeling) after application.

It may be used as a daily facial cleanser, a makeup remover, or a shampoo. The composition of the present invention is a rinse-off product. Thus, such a composition can be applied on the skin (i.e. face and/or body), or the hair, and then rinsed with flush water.

According to another aspect, the present invention provides a non-therapeutic process for cleansing and/or removing makeups from keratin materials, in particular the skin or the hair, comprising the application to the keratin materials, in particular the skin or the hair, of the composition according to the present invention, and rinsing off said composition after an optional period of time.

Other subjects and characteristics, aspects and advantages of the invention will emerge even more clearly on reading the description and the examples that follows.

### DETAILED DESCRIPTION OF THE INVENTION

Unless otherwise defined, all technical and scientific terms used herein have the same meaning as commonly understood by those skilled in the art the present invention belongs to. When the definition of a term in the present description conflicts with the meaning as commonly understood by those skilled in the art the present invention belongs to, the definition described herein shall apply.

In that which follows and unless otherwise indicated, the limits of a range of values are included within this range, in particular in the expressions “between . . . and . . . ” and “from . . . to . . . ”.

Moreover, the expression “at least one” used in the present description is equivalent to the expression “one or more”.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

Unless otherwise specified, all numerical values expressing amount of ingredients and the like which are used in the description and claims are to be understood as being modified by the term “about”. Accordingly, unless indicated to the contrary, the numerical values and parameters described herein are approximate values, which are capable of being changed according to the desired purpose as required.

For the purposes of the present invention, the term “keratin material” is intended to cover human skin, mucous membranes such as the lips, and the hair. Facial skin and the hair are most particularly considered according to the present invention.

All percentages in the present invention refer to weight percentage, unless otherwise specified.

According to an aspect, the present invention relates to a composition for cleansing and/or removing makeups from keratin materials, comprising, in an aqueous phase:
a) at least one amino acid surfactant;
b) at least one C12-C24 saturated monoalcohol; and
c) at least one divalent and/or trivalent metal salt.

### Amino Acid Surfactants

The composition according to the present invention comprises at least one amino acid surfactant.

In one embodiment, said amino acid surfactant is derived from a carboxylate salt of amino acid wherein the amino group situated on the α-carbon or β-carbon of an amino acid salt is acylated with a C₈-C₂₂ fatty acid derivative.

The carboxylate salts of these amino acids can be formed by conventional means such as by neutralization of the respective amino acid with a base. The amino group situated on the α-carbon or β-carbon of the neutralized amino acid is acylated with a fatty acid halide (acyl halide) in the presence of a base via the well-known Schotten-Baumann reaction giving the amide, thus forming the desired surfactant reaction product, i.e. the amino acid surfactant. Suitable acyl halides for acylation of the amino acid carboxylate salt include acyl chlorides, bromides, fluorides, and iodides. The acyl halides can be prepared by reacting a saturated or unsaturated, linear or branched C₈-C₂₂ fatty acid with a thionyl halide (bromide, chloride, fluoride, and iodide). Representative acyl halides include but are not limited to the acyl chlorides selected from decanoyl chloride, dodecanoyl chloride (lauroyl chloride), cocoyl chloride (coconut oil derived fatty acid chlorides) tetradecanoyl chloride (myristoyl chloride), hexadecanoyl chloride (palmitoyl chloride), octadecanoyl chloride (stearoyl chloride), 9-octadecenoyl chloride (oleoyl chloride), eicosanoyl chloride (arachidoyl chloride), docosanoyl chloride (behenoyl chloride), and any mixture thereof. Other acyl halides include the bromides, fluorides and iodides of the foregoing fatty acids. A method for preparing acyl halides as well as an alternative method for acylating amino acids is set forth in US Patent Application Publication No. 2008/0200704, published on Aug. 21, 2008, which application is incorporated herein by reference.

Preferably, said amino acid surfactant is represented by the formula (I):

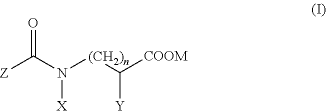

wherein:
Z represents a saturated or unsaturated, linear or branched hydrocarbon group having 8 to 22 carbon atoms,
X is hydrogen or methyl group,
n is 0 or 1,
Y is selected from hydrogen, —CH₃, —CH(CH₃)₂, —CH₂CH(CH₃)₂, —CH(CH₃)CH₂CH₃, —CH₂C₆H₅, —CH₂C₂H₄OH, —CH₂OH, —CH(OH)CH₃, —(CH₂)₄NH₂, —(CH₂)₃NHC(NH)NH₂, —CH₂C(O)OM⁺, —(CH₂)₂C(O)OH, —(CH₂)₂C(O)OM⁺, and
M is a salt-forming cation wherein COO is the counter-anion, such as for example sodium, potassium, ammonium, or triethanolamine.

In one embodiment, in formula (I):
Z represents a saturated or unsaturated, linear or branched C₈-C₂₂ alkyl group,
X is a hydrogen or methyl group,
n is 0,
Y is selected from hydrogen, —(CH₂)₂C(O)OH, —(CH₂)₂C(O)O M⁺, and
M is a salt-forming cation wherein COO is the counter-anion, such as sodium, potassium, ammonium, or triethanolamine.

According to a preferred embodiment of the invention, in formula (I):
Z represents a saturated or unsaturated, linear or branched C₈-C₂₂ alkyl group,
X is a hydrogen or methyl group,
n is 0,
Y is selected from hydrogen, —(CH₂)₂C(O)OH, —(CH₂)₂C(O)O M⁺, and
M is a salt-forming cation wherein COO is the counter-anion, such as for example sodium, potassium, ammonium, or triethanolamine.

Examples of the amino acid surfactants are salt of alanine, arginine, aspartic acid, glutamic acid, glycine, isoleucine, leucine, lysine, phenylalanine, serine, tyrosine, valine, sarcosine, and any mixture thereof. More specifically, mentions can be made of the amino acid surfactants such as dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, cocoyl methyl β-alaninate, lauroyl β-alaninate, lauroyl methyl β-alaninate, myristoyl β-alaninate, potassium lauroyl methyl β-alaninate, sodium cocoyl alaninate, sodium cocoyl methyl β-alaninate and sodium myristoyl methyl β-alaninate palmitoyl glycinate, sodium lauroyl glycinate, sodium cocoyl glycinate, sodium myristoyl glycinate, potassium lauroyl glycinate, potassium cocoyl glycinate, potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate ammonium lauroyl sarcosinate, sodium lauroyl aspartate, sodium myristoyl aspartate, sodium cocoyl aspartate, sodium caproyl aspartate, disodium lauroyl aspartate, disodium myristoyl aspartate, disodium cocoyl aspartate, disodium caproyl aspartate, potassium lauroyl aspartate, potassium myristoyl aspartate, potassium cocoyl aspartate, potassium caproyl aspartate, dipotassium lauroyl aspartate, dipotassium myristoyl aspartate, dipotassium cocoyl aspartate, dipotassium caproyl aspartate, and mixtures thereof.

References can be made to the commercially available amino acid surfactant of, for example, acylsarcosinates, for instance the sodium lauroyl sarcosinate sold under the name Sarkosyl NL 97 by the company Ciba or sold under the name Oramix L 300 by the company SEPPIC, the sodium myristoyl sarcosinate sold under the name Nikkol Sarcosinate MN® by the company Nikkol or the sodium palmitoyl sarcosinate sold under the name Nikkol Sarcosinate PN® by the company Nikkol; alaninates, for instance the sodium N-lauroyl-N-methylamidopropionate sold under the name Sodium Nikkol Alaninate LN 300 by the company Nikkol or sold under the name Alanone ALER by the company Kawaken, and the N-lauroyl-N-methylalanine triethanolamine sold under the name Alanone Alta® by the company Kawaken; N-acylglutamates, for instance the triethanolamine monococoylglutamate sold under the name Acylglutamate CT-12® by the company Ajinomoto and the triethanolamine lauroylglutamate sold under the name Acylglutamate LT-12® by the company Ajinomoto; glycinates, for instance sodium N-cocoylglycinate sold under the name Amilite GCS-12® by the company Ajinomoto; aspartates, for instance the mixture of triethanolamine N-lauroyl aspartate and of triethanolamine N-myristoylaspartate, sold under the name Asparack by the company Mitsubishi; citrates, and any mixture thereof.

According to the present invention, the preferred amino acid surfactant is selected from sodium lauroyl sarcosinate, sodium cocoyl glycinate, sodium cocoyl glutamate, sodium stearoyl glutamate, disodium cocoyl glutamate, sodium lauroyl glutamate, potassium cocoyl glycinate, TEA-cocoyl glutamate, or a mixture thereof.

Mentions of the preferred amino acid surfactant in the composition which is commercially available can be made to sodium lauroyl sarcosinate (ORAMIX L 30 sold by Seppic), sodium cocoyl glycinate (and) water (AMILITE® GCS-12K sold by Ajinomoto), sodium cocoyl glutamate (and) disodium cocoyl glutamate (AMISOFT® CS-22 sold by Ajinomoto), and sodium lauroyl glutamate (AMISOFT® LS-11 sold by Ajinomoto).

According to a preferred embodiment, the amino acid surfactant is selected from sodium lauroyl sarcosinate, sodium cocoyl glycinate, sodium cocoyl glutamate, sodium stearoyl glutamate, disodium cocoyl glutamate, sodium lauroyl glutamate, potassium cocoyl glycinate, TEA-cocoyl glutamate, and a mixture thereof.

Due to the use of amino acid surfactant, the composition according to the present invention brings no irritation to keratin materials.

Advantageously, the amino acid surfactant is present in the composition according to the present invention in an amount ranging from 3 wt. % to 50 wt. %, preferably from 5 wt. % to 30 wt. %, more preferably from 10 wt. % to 20 wt. % relative to the total weight of the composition.

C₁₂-C₂₄ Saturated Monoalcohols

According to the first aspect, the composition according to the present invention comprises at least one C₁₂-C₂₄ saturated monoalcohol.

The C₁₂-C₂₄ saturated monoalcohol is of structure R—OH with R denoting a alkyl group comprising from 12 to 24 carbon atoms.

Preferably, the monoalcohols comprise from 12 to 22 carbon atoms.

Preferably, the C₁₂-C₂₄ saturated monoalcohol that are suitable for use in the present invention are selected from cetyl alcohol, stearyl alcohol, cetearyl alcohol, myristyl alcohol, lauryl alcohol, tridecyl alcohol, pentadecyl alcohol, hexadecyl alcohol, arachidyl alcohol, and behenyl alcohol; they are preferably selected from lauryl alcohol, behenyl alcohol, cetyl alcohol, stearyl alcohol, and cetearyl alcohol.

As oleyl alcohol that may be most particularly suitable for use in the present invention, use may be made, for example, of the products sold under the name HD OCENOL 80/85 V/MB by the company BASF or CRODACOL A 10 by the company CRODA.

As cetyl alcohols that may be most particularly suitable for use in the present invention, use may be made, for example, of the products sold under the names Ecorol® 16/98 F and Ecorol® 16/98 P sold by the company Ecogreen Oleochemicals, Tegoalkanol® 16 sold by the company Evonik Goldschmidt, Lanette 16 sold by the company Cognis, Vegarol® 1698 sold by the company VVF, Alkonat® 1698 P sold by the company Oxiteno, Cetyl Alcohol 98% Min sold by the company Emery Oleochemicals, Ginol® 16 (98%) sold by the company Godrej Industries, Nacol® 16-98 sold by the company Sasol, Kalcol® 6098 sold by the company Kao, and Acilol® 16 sold by the company Aegis Chemical.

As stearyl alcohols that are most particularly suitable for use in the present invention, use may be made, for example, of those sold under the names Tegoalkanol® 18 sold by the company Evonik Goldschmidt, Ecorol® 18/98 F and Ecorol® 18/98 P sold by the company Ecogreen Oleochemicals, Lanette 18 sold by the company Cognis, Kalcol® 8098 sold by the company Kao, Acilol® 18 sold by the company Aegis Chemical, Nacol® 18-98 sold by the company Sasol and NAA® 45 sold by the company Nihon Yushi.

As cetylstearyl alcohols that are most particularly suitable for use in the present invention, use may be made, for example, of those sold under the names Ecorol® 68/50 F and Ecorol® 68/50 P sold by the company Ecogreen Oleochemicals, Lanette® O OR and Lanette® O OR Flakes sold by the company Cognis, Alkonat® 1618 C50 P sold by the company Oxiteno, Nafol® 16-18 EN sold by the company Sasol, Alcohol Cetoestearilico 50/50 sold by the company Industria Quimica Del Centro, Conol® 30 CK sold by the company New Japan Chemical, Cetylstearyl Alcohol 50:50 sold by the company Evonik Goldschmidt, Kalcol® 6850 sold by the company Kao, Vegarol® 1618 (50:50) sold by the company VVF and Ginol® 1618 50:50 OR sold by the company Godrej Industries.

As behenyl alcohol that may be most particularly suitable for use in the present invention, use may be made, for example, of the products sold under the name LANETTE 22 by the company BASF, under the name VEGAROL 2270 by the company VVF, and under the name NAFOL 1822 C by the company SASOL.

Advantageously, the C₁₂-C₂₄ saturated monoalcohol is present in the composition in an amount ranging from 0.3 wt. % to 10 wt. %, preferably from 0.5 wt. % to 8 wt. %, more preferably from 0.8 wt. % to 5 wt. %, relative to the total weight of the composition.

### Metal Salts

The composition according to the present invention comprises at least one divalent and/or trivalent metal salt.

The divalent and/or trivalent metal salt comprises a metal ion M1ⁿ⁺. Preferably, M1ⁿ⁺ is selected from Mg²⁺, Ca²⁺, Zn²⁺, Fe²⁺, Mn²⁺, Cu²⁺, Al³⁺ and Fe³⁺. The metal salt is selected from metal salts of an organic acid or an inorganic acid.

As example of organic acid, mention can be made of ascorbic acid, formic acid, acetic acid, glycolic acid, gluconic acid, lactic acid, mandelic acid, oxalic acid, maleic acid, malonic acid, glyoxylic acid, succinic acid, adipic acid, fumaric acid, sebacic acid, citric acid, tartaric acid, malic acid, tricarboxylic acid, glutaric acid, glucaric acid, pyrrolidone carboxylic acid, phenol sulfonic acid, salicylic acid, etc.

As example of inorganic acid, mention can be made of sulfuric acid, carbonic acid, silicic acid, hydrochloric acid, nitric acid, phosphoric acid, etc.

Preferably, metal salts of an inorganic acid are selected from metal chlorides, sulfates, nitrates, carbonates and hydrogen carbonates, phosphates, silicates, and mixtures thereof, wherein the metal is selected from Mg, Ca, Zn, Mn, Cu, Al and Fe.

More preferably, metal salts of an inorganic acid are selected from calcium chloride, calcium sulfate, calcium nitrate, calcium carbonate and hydrogen carbonate, calcium phosphate, zinc chloride, zinc sulfate, zinc nitrate, zinc carbonate and hydrogen carbonate, zinc phosphate, magnesium chloride, magnesium sulfate, magnesium nitrate, magnesium carbonate and hydrogen carbonate, magnesium phosphate, magnesium aluminum silicate, aluminum calcium sodium silicate, and mixtures thereof.

Preferably, metal salts of an organic acid are selected from metal ascorbates, formates, acetates, glycolates, gluconates, lactates, mandelates, oxalates, maleates, malonates, glyoxylates, succinates, adipates, fumarates, sebacates, citrates, tartarates, malates, tricarboxylates, glutarates, glucarates, pyrrolidone carboxylates, phenolsulfonate, salicylates, and mixtures thereof, wherein the metal is selected from Mg, Ca, Zn, Mn, Cu, Al and Fe.

More preferably, metal salts of an organic acid are selected from magnesium gluconate, magnesium PCA (Magnesium pyrrolidone carboxylate), magnesium acetate, calcium PCA(calcium pyrrolidone carboxylate), zinc lactate, zinc gluconate, zinc phenolsulfonate, zinc salicylate, Zinc PCA (zinc pyrrolidone carboxylate), zinc citrate, zinc ascorbate, copper PCA(copper pyrrolidone carboxylate), copper gluconate, and mixtures thereof.

According to a preferred embodiment, the metal salt is selected from magnesium gluconate, magnesium PCA, magnesium sulfate, magnesium acetate, magnesium carbonate, magnesium aluminum silicate, calcium carbonate, calcium PCA, aluminum calcium sodium silicate, calcium chloride, calcium carbonate, zinc PCA, zinc gluconate, copper PCA, copper gluconate, and a mixture thereof.

Advantageously, the metal salt is present in the composition according to the present invention in an amount ranging from 0.25 wt. % to 10 wt. %, preferably from 0.25 wt. % to 5 wt. %, more preferably from 0.5 wt. % to 3 wt. %, relative to the total weight of the composition.

### Amphoteric Surfactants

Preferably, the composition according to the present invention further comprises at least one amphoteric surfactant selected from betaines, (C₈-C₂₀)alkyl betaines, (C₈-C₂₀)alkylamido(C₁-C₆)alkylbetaines, and mixtures thereof.

Mention may in particular be made, as betaines, of (C₈-C₂₀)alkyl betaines, such as, coco betaine, such as the product sold under the name Dehyton AB-30® by the company Cognis, lauryl betaine, such as the product sold under the name Genagen KB® by the company Clariant.

Mention may be made, among (C₈-C₂₀)alkylamido(C₁-C₆)alkylbetaines, for example, of cocamidopropyl betaine, sold under the name Lebon 2000 HG® by the company Sanyo or sold under the name Empigen BB® by the company Albright &Wilson, or lauramidopropyl betaine, sold under the name Rewoteric AMB12 Po by the company Witco.

Preferably, the amphoteric surfactant is selected from (C₈-C₂₀)alkylamido(C₁-C₆)alkylbetaines, and more preferably from (C₁₂-C₂₀)alkylamido(C₁-C₆)alkylbetaines. Most preferably, the amphoteric surfactant is cocamidopropyl betaine.

Advantageously, the amphoteric surfactant is present in the composition according to the present invention in an amount ranging from 0.5 wt. % to 10 wt. %, preferably from 0.25 wt. % to 8 wt. %, more preferably from 0.5 wt. % to 5 wt. %, relative to the total weight of the composition.

### Aqueous Phase

The composition of the present invention comprises at least one aqueous phase.

According to a preferred embodiment, the composition of the present invention is an aqueous paste.

The aqueous phase of the composition according to the present invention comprises water and optionally one or more water-miscible or at least partially water-miscible compounds, for instance C₂-C₈ polyols or monoalcohols, such as ethanol and isopropanol.

The term “polyol” should be understood as meaning any organic molecule comprising at least two free hydroxyl groups. Examples of polyols that may be mentioned include glycols, for instance butylene glycol, propylene glycol, dipropylene glycol, isoprene glycol, hexylene glycol, caprylyl glycol, glycerol (i.e. glycerin) and polyethylene glycols.

According to a preferred embodiment, the composition according to the present application further comprises at least one C₂-C₈ polyol selected from butylene glycol, propylene glycol, dipropylene glcol, isoprene glycol, hexylene glycol, caprylyl glycol, glycerin and polyethylene glycols.

With the presence of C₂-C₈ polyol, it is easy to form a crystalized system. Preferably, the C₂-C₈ polyol is present in an amount ranging from 30 wt. % to 40 wt. %, relative to the total weight of the composition.

The aqueous phase may represent from 40 wt. % to 70 wt. %, preferably from 45 wt. % to 65 wt. %, relative to the total weight of the composition.

### Additional Ingredients

The composition according to the present invention may comprise one or more additional ingredients, selected from those conventionally used in skincare cleasners.

The composition in accordance with the present invention may comprise any of the following additives: pH adjusting agents (e.g. citric acid); additional surfactants; biological extracts; antibacterial agents, fragrances; thickeners; and cationic preservatives.

A person skilled in the art can adjust the type and amount of additional ingredients present in the compositions according to the present invention by means of routine operations, so that the desired properties of these compositions are not adversely affected by the additional ingredients.

According to a preferred embodiment, the present invention relates to a composition for cleansing and/or removing makeups from keratin materials, comprising in an aqueous phase, relative to the total weight of the composition:
a) from 10 wt. % to 20 wt. % of at least one amino acid surfactant selected from sodium lauroyl sarcosinate, sodium cocoyl glycinate, sodium cocoyl glutamate, sodium stearoyl glutamate, disodium cocoyl glutamate, sodium lauroyl glutamate, potassium cocoyl glycinate, TEA-cocoyl glutamate, and a mixture thereof;
b) from 0.8 wt. % to 5 wt. % of at least one C₁₂-C₂₄ saturated monoalcohol selected from;
c) from 0.5 wt. % to 3 wt. % of at least one divalent and/or trivalent metal salt selected from magnesium gluconate, magnesium PCA, magnesium sulfate, magnesium acetate, magnesium carbonate, magnesium aluminum silicate, calcium carbonate, calcium PCA, aluminum calcium sodium silicate, calcium chloride, calcium carbonate, zinc PCA, zinc gluconate, copper PCA, copper gluconate, and a mixture thereof; and
d) from 30 wt. % to 40 wt. % of at least one C₂-C₈ polyol selected from butylene glycol, propylene glycol, dipropylene glycol, isoprene glycol, hexylene glycol, caprylyl glycol, glycerin, and polyethylene glycols.

### Process and Use

The composition according to the present invention can be used in a process for cleansing and/or removing makeups from keratin materials, such as the skin, in particular the face, by being applied to the keratin materials.

The composition according to the present invention is a crystalized system in the form of a paste, a cream or a gel textured lotion.

The composition according to the present invention is mild and oil-resistant, and does not have a dripping issue.

It was found that the composition according to the present invention can provides a good foam density, a quick rinse-off speed, and a good skin finish (i.e. a not tight and not dry skin feeling) after application.

The composition according to the invention may be applied by any means enabling a uniform distribution, in particular using a finger, or a cotton ball, and can be removed by rinsing with water.

Thus, according to another aspect, the present invention relates to a non-therapeutic process for cleansing and/or removing makeups from keratin materials, in particular the skin, comprising the application to the keratin materials, in particular the skin, of the composition according to the present invention, and rinsing off said composition after an optional period of time.

The present invention is illustrated in greater detail by the examples described below, which are given as non-limiting illustrations.

### EXAMPLES

Main raw materials used, trade names and supplier thereof are listed in Table 1.

TABLE 1

| INCI Name | Trade Name | Supplier |
|---|---|---|
| SODIUM COCOYL GLYCINATE | AMILITE GCS-12K | AJINOMOTO |
| CITRIC ACID | CITRIC ACID MONOHYDRATE | CITRIQUE BELGE |
| | GRANULAR | |
| MAGNESIUM GLUCONATE | GLUCONAL ® MG-P | PURAC |
| LAURYL ALCOHOL | VEGAROL 1216(MB) | VVF |
| OLEYL ALCOHOL | HD OCENOL 80/85 V/MB | BASF |
| CETYL ALCOHOL | LANETTE 16 | BASF |
| STEARYL ALCOHOL | LANETTE 18 | BASF |
| BEHENYL ALCOHOL | LANETTE 22 | BASF |
| COCAMIDOPROPYL BETAINE | DEHYTON ® PK 45 | BASF |
| SODIUM COCOYL GLYCINATE | AMILITE GCS-12K | AJINOMOTO |

### Comparative Example 1-3 and Invention Examples 1-9

The composition according to comparative examples (CE) 1-3 (CE) and invention examples (IE) 1-9 comprising the ingredients shown in Table 2 were prepared, with all amounts expressed by percentages by weight of active matter with regard to the total weight of each composition.

### Preparation Process

The compositions were prepared as follows, taking the composition of invention example 1 as an example.

Sodium cocoyl glycinate and glycerin were introduced into a main kettle and heated to 80° ° C. with stirring. Then water and citric acid were pre-mixed to obtain a mixture and the mixture was introduced into the main kettle with stirring. Next, magnesium gluconate, lauryl alcohol, and cocamidopropyl betaine were introduced in the main kettle one by one with stirring, to obtain the composition, which was then cooled down to room temperature.

### Evaluation of Compositions

The compositions prepared were evaluated in terms of texture, rinse-off speed, foaming density, and oil resistance.

### Texture

It was observed that the compositions of invention examples 1-9 are in the form of cream, and there is no dripping issue.

### Rinse-Off Speed

The rinse-off speed is used to define quickness of the strongest squeaky feeling of whole face occurs and was tested as follows:
a) Rinse hands under tap water for 2 seconds, move the wet hands (do not reserve water in palms) on the face from the chin to the forehead and back to the chin (2 sides of the face);
b) Repeat this rinsing procedure 15 times; and
c) Dry hands with a dry towel.

Speed of squeaky feel occurrence was evaluated tactilely during 15 rinsing movements. A score within a range of 1-15 was given, wherein 1-4 means a low rinse-off speed, 5-9 means a middle rinse-off speed and 10-15 means a high rinse-off speed.

### Foaming Density

Foam density indicates the compactness of foam quality.

Preparation: taking 0.5 ml of the composition to be tested with a syringe, 3×1 ml of water with pipettes.

### Procedure:

1) Rinse hands under tap water for 2 seconds and place the 0.5 ml of the composition to be tested to palm with the syringe, add 1 ml of water with one pipette;
2) Make back and forth movements for 20 rounds (2 circles/second) and pause to gather foam in the palm to prevent foam from flowing if necessary;
3) Add 1 ml of water with one pipette again, and make back and forth movements for 20 rounds (2 circles/second), and pause to gather foam in the palm to prevent foam from flowing if necessary
4) Put together all the foam in one palm.

A score within a range of 1-15 was given, wherein 1-4 means a low foam density, 5-9 means a middle foam density and 10-15 means a high foam density.

### Foaming Volume and Oil Resistance

The foaming density was determined by dissolving 20 g sample in 80 g of tab water with or without 0.7 g artificial sebum with the following composition in Table 2, stirring with a Kitchen Blender for 2 minutes, and then measuring the foam volume with a cylinder. Each sample was tested for 3 times and the results were averaged.

TABLE 2

| | Trade Name | Supplier | Content(wt. %) |
|---|---|---|---|
| OLEIC ACID | WILFARIN OA-7075 | WILMAR | 13.8 |
| TRIISOSTEARIN | TRIISOSTEARATE DE GLYCEROL | STEARINERIE | 21.4 |
| | | DUBOIS | |
| CHOLESTEROL | CHOLESTEROL NM | WILMAR | 5.8 |
| CETYL PALMITATE | CUTINA CP | BASF | 24.2 |
| STEARIC ACID | PALMERA B1802CG MB | KLK OLEO | 13.8 |
| SQUALANE | PLANTASENS OLIVE SQUALANE | CLARIANT | 10.3 |
| GLYCERYL OLEATE | MONOMULS 90-O 18 | BASF | 10.7 |

The oil resistance was determined by calculating the volume decrease percentage (Vdescrease %) according to the following formula:

Vdescrease %=[V(without sebum)−V(with sebum)]/V(without sebum)*100%

If the volume decrease percentage is in the range of 30%-100%, it means the oil resistance is poor. If the volume decrease percentage is in the range of 0-15%, it means the oil resistance is good.

The results obtained were also summarized in Table 3.

TABLE 3

| | CE1 | CE 2 | CE 3 | IE1 | IE2 | IE3 | IE4 | IE5 | IE 6 | IE 7 | IE 8 | IE9 |
|---|---|---|---|---|---|---|---|---|---|---|---|---|
| SODIUM COCOYL | 13 | 13 | 13 | 13 | 13 | 13 | 13 | 13 | 13 | 13 | 13 | 14 |
| GLYCINATE | | | | | | | | | | | | |
| GLYCERIN | 39.3 | 39.3 | 39.3 | 39.3 | 39.3 | 39.3 | 39.3 | 39.3 | 39.3 | 39.3 | 39.3 | 39.3 |
| WATER | QS100 | QS100 | QS100 | QS100 | QS100 | QS100 | QS100 | QS100 | QS100 | QS100 | QS100 | QS100 |
| CITRIC ACID | PH = 6.1 | PH = 6.1 | PH = 6.1 | PH = 6.1 | PH = 6.1 | PH = 6.1 | PH = 6.1 | PH = 6.1 | PH = 6.1 | PH = 6.1 | PH = 6.1 | PH = 6.1 |
| MAGNESIUM | — | 1 | — | 1 | 1 | 1 | 1 | 1 | 1 | 1 | 1 | 1 |
| GLUCONATE | | | | | | | | | | | | |
| LAURYL ALCOHOL | — | — | — | 3 | — | — | — | — | 1.5 | 1.5 | — | — |
| CETYL ALCOHOL | — | — | — | — | 3 | — | — | — | 1.5 | — | 1 | 2 |
| OLEYL ALCOHOL | — | — | — | — | — | 3 | — | — | — | — | — | — |
| STEARYL | — | — | 3 | — | — | — | 3 | — | — | — | — | — |
| ALCOHOL | | | | | | | | | | | | |
| BEHENYL | — | — | — | — | — | — | — | 3 | — | 1.5 | 2 | 1 |
| ALCOHOL | | | | | | | | | | | | |
| COCAMIDOPROPYL | — | 1 | 1 | 1 | 1 | 1 | 1 | 1 | 1 | 1 | 1 | — |
| BETAINE | | | | | | | | | | | | |
| *Rinse-off | 8 | 12 | 8 | 12 | 12 | 12 | 12 | 12 | 12 | 12 | 12 | 12 |
| *Foaming density | 5 | 8 | 10 | 11 | 11 | 10 | 10 | 11 | 13 | 13 | 14 | 11 |
| Foam Volume | | 843 ml | | 870 | 900 ml | | | 880 ml | 830 ml | 830 ml | 890 ml | 830 ml |
| without sebum | | | | | | | | | | | | |
| Foam Volume | | 547 ml | | 780 | 820 ml | | | 800 ml | 750 ml | 760 ml | 823 ml | 750 ml |
| with sebum | | | | | | | | | | | | |
| Volme decrease | | −35% | | −10% | −9% | | | −9% | −10% | −8% | −7% | −9% |
| percentage (%) | | | | | | | | | | | | |

*Max = 15, the higher, the better.

It can be seen from Table 3, as compared with the composition of comparative example 1, the compositions of invention examples 1-9 show better foam density.

As compared with the composition of comparative examples 2 and 3, the compositions of invention examples 1-9 show better rinse off property.

The compositions of invention examples 1-2 and 5-9 demonstrate good oil resistance.

During the test of rinse-off speed, it was found that compositions of invention examples 1-9 deliver a good skin finish (not tight and not dry skin feeling), and brings no irritation to the face.

1. A composition for cleansing and/or removing makeups from keratin materials, comprising in an aqueous phase:
a) at least one amino acid surfactant;
b) at least one C₁₂-C₂₄ saturated monoalcohol; and
c) at least one divalent and/or trivalent metal salt.

2. The composition according to claim 1, wherein the amino acid surfactant is represented by the formula (I): 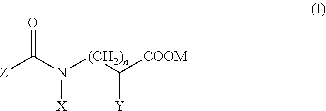
wherein:
Z represents a saturated or unsaturated, linear or branched hydrocarbon group having
8 to 22 carbon atoms,
X is hydrogen or methyl group,
n is 0 or 1,
Y is selected from hydrogen, —CH₃, —CH (CH₃)₂, —CH₂CH (CH₃)₂, —CH (CH₃) CH₂CH₃,
—CH₂C₆H₅, —CH₂C₂H₄OH, —CH₂OH, —CH (OH) CH₃, —(CH₂)₄NH₂, —(CH₂)₃NHC (NH) NH2, —CH₂C (O)O-M⁺, —(CH₂)₂C (O) OH, —(CH₂)₂C (O)O—M⁺, and
M is a salt-forming cation wherein COO is the counter-anion
in formula (I):
Z represents a saturated or unsaturated, linear or branched C₈-C₂₂alkyl group,
X is a hydrogen or methyl group,
n is 0,
Y is selected from hydrogen, —(CH₂)>C (O) OH, —(CH₂)¿C (O)O—M⁺, and
M is a salt-forming cation wherein COO is the counter-anion.

3. The composition according to claim 1, wherein the amino acid surfactant is selected from the group consisting of sodium lauroyl sarcosinate, sodium cocoyl glycinate, sodium cocoyl glutamate, sodium stearoyl glutamate, disodium cocoyl glutamate, sodium lauroyl glutamate, potassium cocoyl glycinate, TEA-cocoyl glutamate, and a mixture thereof.

4. The composition according to claim 1, wherein the amino acid surfactant is present in an amount ranging from 3 wt. % to 50 wt. %, relative to the total weight of the composition.

5. The composition according to claim 1, wherein the C₁₂-C₂₄ saturated monoalcohol is selected from the group consisting of cetyl alcohol, stearyl alcohol, cetearyl alcohol, myristyl alcohol, lauryl alcohol, tridecyl alcohol, pentadecyl alcohol, hexadecyl alcohol, arachidyl alcohol, and behenyl alcohol.

6. The composition according to claim 1, wherein the C₁₂-C₂₄ saturated monoalcohol is present in an amount ranging from 0.3 wt. % to 10 wt %, relative to the total weight of the composition.

7. The composition according to claim 1, wherein the metal salt comprises a metal ion M1ⁿ⁺ selected from the group consisting of Mg²⁺, Ca²⁺, Zn²⁺, Fe²⁺, Mn²⁺, Cu²⁺, Al³⁺ and Fe³⁺.

8. The composition according to claim 1, wherein the metal salt is present in an amount ranging from 0.25 wt. % to 10 wt. %, relative to the total weight of the composition.

9. The composition according to claim 1, further comprising at least one amphoteric surfactant selected from the group consisting of (C₁₂-C₂₀) alkylamido (C₁-C₆) alkylbetaines.

10. The composition according to claim 9, wherein the amphoteric surfactant is present in an amount ranging from 0.5 wt. % to 10 wt. %, relative to the total weight of the composition.

11. The composition according to claim 1, further comprising at least one C₂-C₈ polyol selected from the group consisting of butylene glycol, propylene glycol, dipropylene glycol, isoprene glycol, hexylene glycol, caprylyl glycol, glycerin and polyethylene glycols.

12. The composition according to claim 11, wherein the C₂-C₈ polyol is present in an amount ranging from 30 wt. % to 40 wt. %, relative to the total weight of the composition.

13. The composition according to claim 1, wherein the aqueous phase represents from 40 wt. % to 70 wt. %, relative to the total weight of the composition.

14. The composition according to claim 1, wherein the composition is a crystalized system in the form of a paste, a cream or a gel textured lotion.

15. A non-therapeutic process for cleansing and/or removing makeups from keratin materials, comprising the application to the keratin materials, of the composition according to claim 1, and rinsing off said composition after an optional period of time.
